(19) United States
(12) Patent Application Publication
(10) Pub. No.: US 2022/0090182 A1
(43) Pub. Date: Mar. 24, 2022

(54) FLUORESCENT IN-SITU HYBRIDIZATION IMAGING USING MULTIPLEXED FLUORESCENT SWITCHING

(71) Applicant: Applied Materials, Inc., Santa Clara, CA (US)

(72) Inventors: Zhen Li, Singapore (SG); Marc Glazer, San Jose, CA (US); Yun-Ching Chang, San Jose, CA (US)

(21) Appl. No.: 17/481,161

(22) Filed: Sep. 21, 2021

Related U.S. Application Data
(60) Provisional application No. 63/081,872, filed on Sep. 22, 2020.

(51) Int. Cl. C12Q 1/6841; C12Q 1/6876; C12Q 1/6825; G01N 21/64

(52) U.S. Cl. CPC C12Q 1/6841; C12Q 1/6876; G01N 2021/6439; G01N 21/6456; G01N 21/6428; C12Q 1/6825

(57) ABSTRACT

A method of fluorescent in-situ hybridization imaging includes exposing a sample to a first plurality of first readout probes and a second plurality of second readout probes, obtaining a first image of the sample with first readout probes and the second readout probes bound to a first analyte and a second analyte, respectively, so as to emit light at a first wavelength range, treating the sample so as to modify the second readout probes, and obtaining a second image of the sample with the first readout probes bound to the first analyte so as to emit light at the first wavelength range and the second analyte substantially not emitting light at the first wavelength range.

### CLAIM OF PRIORITY

This application claims priority under 35 USC § 119(e) to U.S. Patent Application Ser. No. 63/081,872, filed on Sep. 22, 2020, the entire contents of which are hereby incorporated by reference.

### TECHNICAL FIELD

This specification relates to increased readout probe combinations in multiplexed fluorescence in-situ hybridization imaging.

### BACKGROUND

It is of great interest to the biotech community and pharmaceutical industry to develop methods for visualizing and quantifying multiple biological analytes—e.g., DNA, RNA, and protein—within a biological sample—e.g., tissue resection, biopsy, cells grown in culture. Scientists use such methods to diagnose/monitor disease, validate biomarkers, and investigate treatment. To date, exemplary methods include multiplex imaging of antibodies or oligonucleotides (e.g., RNA or DNA) labeled with a functional domain to a biological sample.

Multiplexed fluorescence in-situ hybridization (mFISH) imaging is a powerful technique to determine gene expression in spatial transcriptomics. In brief, a sample is exposed to multiple oligonucleotide probes that target RNA of interest. These probes have different labeling schemes that will allow one to distinguish different RNA species when the complementary, fluorescent labeled probes are introduced to the sample. Sequential rounds of fluorescence images are then acquired with exposure to excitation light of different wavelengths. For each given pixel, its fluorescence intensities from the different images for the different wavelengths of excitation light form a signal sequence. This sequence is then compared to a library of reference codes from a codebook that associates each code with a gene. The best matching reference code is used to identify an associated gene that is expressed at that pixel in the image.

### SUMMARY

In one aspect, a method of fluorescent in-situ hybridization imaging includes exposing a sample to a plurality of encoding probes, exposing the sample to a first plurality of first readout probes and a second plurality of second readout probes, obtaining a first image of the sample with the first encoding probes and the second encoding probes having the first readout probes and the second readout probes, respectively, bound thereto so as to emit light at the first wavelength range, treating the sample so as to modify the second readout probes on the second plurality of second encoding probes, and obtaining a second image of the sample with the first encoding probes having the first readout probes bound thereto so as to emit light at the first wavelength range and the second encoding probes substantially not emitting light in at the first wavelength range. Each encoding probe of the plurality of encoding probes has an encoding portion that targets a nucleotide sequence in the sample and a hybridization portion. Each first readout probe of the first plurality of first readout probes has a fluorophore to emit light at a first wavelength range and a first targeting portion that binds to a first hybridization sequence in a first encoding probe of the plurality of encoding probes, and each second readout probe of the second plurality of second readout probes has a fluorophore to emit light at the first wavelength range and a second targeting portion that binds to a second hybridization sequence in a second encoding probe of the plurality of encoding probes.

Advantages of implementations can include, but are not limited to, one or more of the following.

In multiplexed fluorescence in-situ hybridization (mFISH) imaging and processing, the information capacity per round of hybridization can be substantially increased. In particular, with these switching modalities capable of switching a readout probe from an on bit to an off bit, or vice versa, additional image layers can be acquired in each round of hybridization without the need for photobleaching or introduction and hybridization of additional encoding probes. This increases the readout call bit depth and imaging throughput.

For example, different nucleotide sequences can be targeted with two unique readout probes sharing the same fluorophore and color channel but having different switching modalities. This allows for different readout calls after a switching step, which can increase the amount of information gained between each hybridization and photobleaching step. This can lead to faster data acquisition and cost savings through increased image acquisition per consumable reagent.

The genetic code book used to identify targeted genes with a string of readout calls can also gain added capacity when each nucleotide sequence can be targeted by switchable readout probes. For example, the maximum target number of a 16 bit code book gene length increases with each switchable readout probe added to a set. In this manner, many more targets can be achieved per assay with the same code book length and the same Hamming distance between code words.

This method is also compatible with existing mFISH imaging systems with any number of color channels. The method increases the bit readout call potential for each channel in a system, regardless of the total number of channels.

The details of one or more embodiments are set forth in the accompanying drawings and the description below. Other features, aspects, and advantages will become apparent from the description, the drawings, and the claims.

### BRIEF DESCRIPTION OF THE DRAWINGS

FIG. 1 is a schematic diagram of an apparatus for multiplexed fluorescence in-situ hybridization imaging.

FIG. 2 is a flow chart of the steps involved in mFISH imaging.

FIGS. 3A-3I are exemplary readout probe designs.

FIG. 4A depicts a readout probe bound to the hybridization region of an encoding probe.

FIG. 4B depicts a readout probe including a switchable modality bound to the hybridization region of an encoding probe.

FIG. 4C depicts of a readout probe including a switchable modality after the cleavage site has been cleaved.

FIG. 5 is a flow chart of the steps involved in mFISH imaging including one or more switching steps.

FIG. 6A depicts an example of readout call depth using three example readout probes.

FIG. 6B depicts an example of readout call depth using three example readout probes and two quenching probes.

FIG. 6C depicts an example of readout call depth using eight readout probes and two fluorophores.

FIG. 7 is a flow chart of a method of data processing.

FIG. 8 illustrates a method of decoding.

FIG. 9 illustrates an example of the method of decoding using two readout probes.

Like reference numbers and designations in the various drawings indicate like elements.

### DETAILED DESCRIPTION

Multiplexed fluorescence in-situ hybridization (mFISH) imaging is a powerful technique that uses fluorescent readout probes that bind to only those parts of an encoding probe that share a high degree of sequence complementarity. This allows numerous nucleic acid species and specific genes to be targeted, imaged, and quantified in single cells in their native context.

Encoding probes can target nucleotide sequences in numerous species of nucleic acid, including DNA, mRNA, lncRNA and miRNA in tissues and cells. The nucleotide sequences can be sequences within a gene or that span multiple genes. mFISH allows pinpointed targeting by creating an encoding probe with a nucleotide encoding sequence that is complimentary to a sequence within the target gene. The 3′ and 5′ regions of the encoding probes do not share sequence specificity with the underlying target nucleotide sequence and instead are engineered hybridization sequences complimentary to nucleotide sequences of readout probes.

mFISH readout probes include a constructed nucleotide sequence in conjunction with a fluorophore. The nucleotide sequence, e.g., targeting sequence, of each readout probe targets and binds to one engineered hybridization region of an encoding probe. A light source then excites the fluorophore and the resultant emitted fluorescent light is imaged with a microscope. Multiple fluorophores that have different excitation wavelengths and/or have different emission wavelengths permit multiple images in different color channels to be acquired following a single round of hybridization of the readout probes.

In order to further increase the number of readout probes without increasing the number of color channels, the readout probes from a previous round of hybridization can be photobleached, a new set of readout probes can be introduced to the sample and hybridized to the encoding probes, and a new round of images can be obtained. The photobleaching, hybridization and imaging steps can be repeated multiple times.

However, steps that occur over long timescales (e.g., >10 minutes) can limit imaging throughput during consecutive rounds of hybridization, imaging, and photobleaching of hybridized readout probes. The total time required for a mFISH measurement is composed of the time required to image the total sample area and the time required to complete the image-independent assay steps, such as the hybridization steps between imaging. For example, high illumination intensities are used to photobleach the fluorescence signals between consecutive rounds of mFISH. Each hybridization probe must be photobleached before a new probe can be added to the sample and each readout probe must be allowed an incubation time to fully hybridize with their target hybridization region.

Disclosed herein is a system in which fluorescent signals from the hybridized readout probes are ‘switched’ and the sample re-imaged before photobleaching. Readout probes are designed such that their fluorescence state can be switched from an ‘on’ to an ‘off’ state, or vice versa, within a ‘switching’ step. By nesting one or more switching steps within a round of hybridization, the average time between imaging steps (e.g., data collection) can be reduced and a further level of data multiplexing can be added for use in identifying gene codes within the code book. Designed readout probes incorporate switchable modalities to be modified between imaging rounds without performing additional hybridization, which is commonly the longest step in an mFISH process.

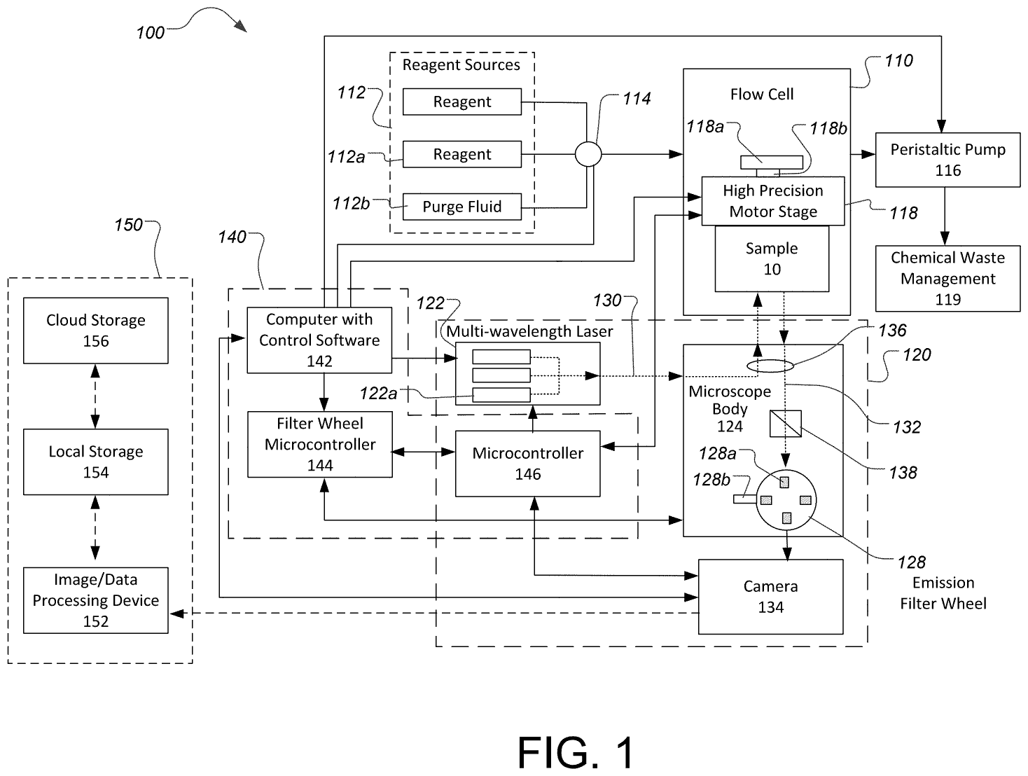

Referring to FIG. 1, a multiplexed fluorescent in-situ hybridization (mFISH) imaging and image processing apparatus 100 includes a flow cell 110 to hold a sample 10, a fluorescence microscope 120 to obtain images of the sample 10, and a control system 140 to control operation of the various components of the mFISH imaging and image processing apparatus 100. The control system 140 can include a computer 142, e.g., having a memory, processor, etc., that executes control software.

The fluorescence microscope 120 includes an excitation light source 122 that can generate excitation light 130 of multiple different wavelengths. In particular, the excitation light source 122 can generate narrow-bandwidth light beams having different wavelengths at different times. For example, the excitation light source 122 can be provided by a multi-wavelength continuous wave laser system, e.g., multiple laser modules 122a that can be independently activated to generate laser beams of different wavelengths. Output from the laser modules 122a can be multiplexed into a common light beam path.

The fluorescence microscope 120 includes a microscope body 124 that includes the various optical components to direct the excitation light from the light source 122 to the flow cell 110. For example, excitation light from the light source 122 can be coupled into a multimode fiber, refocused and expanded by a set of lenses, then directed into the sample by a core imaging component, such as a high numerical aperture (NA) objective lens 136. When the excitation channel needs to be switched, one of the multiple laser modules 122a can be deactivated and another laser module 122a can be activated, with synchronization among the devices accomplished by one or more microcontrollers 144, 146.

The objective lens 136, or the entire microscope body 124, can be installed on a vertically movable mount coupled to a Z-drive actuator. Adjustment of the Z-position, e.g., by a microcontroller 146 controlling the Z-drive actuator, can enable fine tuning of focal position. Alternatively, or in addition, the flow cell 110 (or a stage 118 supporting the sample in the flow cell 110) could be vertically movable by a Z-drive actuator 118b, e.g., an axial piezo stage. Such a piezo stage can permit precise and swift multi-plane image acquisition.

The sample 10 to be imaged is positioned in the flow cell 110. The flow cell 110 can be a chamber with cross-sectional area (parallel to the object or image plane of the microscope) with and area of about 2 cm by 2 cm. The sample 10 can be supported on a stage 118 within the flow cell, and the stage (or the entire flow cell) can be laterally movable, e.g., by a pair of linear actuators 118a to permit XY motion. This permits acquisition of images of the sample 10 in different laterally offset fields of view (FOVs). Alternatively, the microscope body 124 could be carried on a laterally movable stage.

An entrance to the flow cell 110 is connected to a set of hybridization reagents sources 112. A multi-valve positioner 114 can be controlled by the controller 140 to switch between sources to select which reagent 112a is supplied to the flow cell 110. Each reagent includes a different set of one or more oligonucleotide probes. Each probe targets a different nucleotide sequence on a different encoding probe (and thus targets a different RNA sequence), and has a different set of one or more fluorescent materials, e.g., phosphors, that are excited by different combinations of wavelengths. In addition to the reagents 112a, there can be a source of a purge fluid 112b, e.g., DI water.

An exit to the flow cell 110 is connected to a pump 116, e.g., a peristaltic pump, which is also controlled by the controller 140 to control flow of liquid, e.g., the reagent or purge fluid, through the flow cell 110. Used solution from the flow cell 110 can be passed by the pump 116 to a chemical waste management subsystem 119.

In operation, the controller 140 causes the light source 122 to emit the excitation light 130, which causes fluorescence of fluorescent material in the sample 10, e.g., fluorescence of the probes that are bound to RNA in the sample and that are excited by the wavelength of the excitation light. The emitted fluorescent light 132, as well as back propagating excitation light, e.g., excitation light scattered from the sample, stage, etc., are collected by an objective lens 136 of the microscope body 124.

The collected light can be filtered by a multi-band dichroic mirror 138 in the microscope body 124 to separate the emitted fluorescent light from the back propagating illumination light, and the emitted fluorescent light is passed to a camera 134. The multi-band dichroic mirror 138 can include a pass band for each emission wavelength expected from the probes under the variety of excitation wavelengths. Use of a single multi-band dichroic mirror (as compared to multiple dichroic mirrors or a movable dichroic mirror) can provide improved system stability.

The camera 134 can be a high resolution (e.g., 2048×2048 pixel) CMOS (e.g., a scientific CMOS) camera, and can be installed at the immediate image plane of the objective. Other camera types, e.g., CCD, may be possible. When triggered by a signal, e.g., from a microcontroller, image data from the camera can be captured, e.g., sent to an image processing system 150. Thus, the camera 134 can collect a sequence of images from the sample.

To further remove residual excitation light and minimize cross talk between excitation channels, each laser emission wavelength can be paired with a corresponding band-pass emission filter 128a. Each filter 128a can have a wavelength of 10-50 nm, e.g., 14-32 nm. In some implementations, a filter is narrower than the bandwidth of the fluorescent material of the probe resulting from the excitation, e.g., if the fluorescent material of the probe has a long trailing spectral profile.

The filters are installed on a high-speed filter wheel 128 that is rotatable by an actuator 128b. The filter wheel 128 can be installed at the optical infinity to minimize optical aberration in the imaging path. After passing the emission filter of the filter wheel 128, the cleaned fluorescence signals can be refocused by a tube lens and captured by the camera 134. The dichroic mirror 138 can be positioned in the light path between the objective lens 136 and the filter wheel 128.

To facilitate high speed, synchronized operation of the system, the control system 140 can include two microcontrollers 144, 146 that are employed to send trigger signals, e.g., TTL signals, to the components of the fluorescence microscope 120 in a coordinated manner. The first microcontroller 144 is directly run by the computer 142, and triggers actuator 128b of the filter wheel 128 to switch emission filters 128a at different color channels. The first microcontroller 144 also triggers the second microcontroller 146, which sends digital signals to the light source 122 in order to control which wavelength of light is passed to the sample 10. For example, the second microcontroller 146 can send on/off signals to the individual laser modules of the light source 122 to control which laser module is active, and thus control which wavelength of light is used for the excitation light. After completion of switching to a new excitation channel, the second microcontroller 146 controls the motor for the piezo stage 118b to select the imaging height. Finally the second microcontroller 146 sends a trigger signal to the camera 134 for image acquisition.

Communication between the computer 142 and the device components of the apparatus 100 is coordinated by the control software. This control software can integrate drivers of all the device components into a single framework, and thus can allow a user to operate the imaging system as a single instrument (instead of having to separately control many devices).

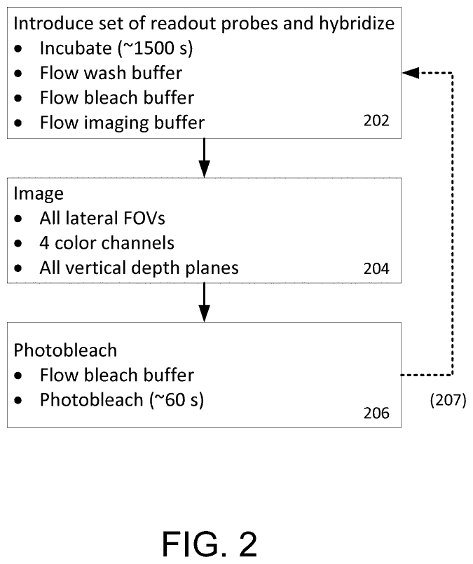

In order to provide context, the conventional mFISH traditional round of mFISH imaging and genetic identification relies on a series of nested steps including hybridization, imaging, and photobleaching. FIG. 2 demonstrates the workflow for a conventional round of mFISH with some timescales included for reference. Prior to mFISH imaging, encoding probes are added to a biological sample containing sequences to be targeted. The target nucleotide sequences are bound with a library of encoding probes, each encoding probe containing an encoding sequence that binds to a specific targeting sequence, and a hybridization region at each end of the encoding sequence. The hybridization regions are designed to bind the targeting sequences present in the set of readout probes, but not to the sequences of the sample.

The first round of hybridization of the readout probes (202) begins with the multi-valve positioner 114 supplying a buffer containing readout probes to the flow cell 110 containing the sample 10. As described above, each readout probe includes a fluorophore coupled to an oligonucleotide targeting sequence designed to bind to one of the hybridization regions of the encoding probes. In practice, there can be multiple groups of readout probes, with readout probes within a group having the same oligonucleotide targeting sequence and the same fluorophore, but readout probes of different groups having oligonucleotide targeting sequences and different fluorophores that emit at different wavelengths. The total number of groups of readout probes can be equal to or less than the number of color channels the system is capable of imaging. For example, a control system 140 with a light source 122 with four laser modules can excite a set of four unique fluorophores in a sequence of four rounds of excitation and imaging.

The system performs an incubation step allowing the set of readout probes to penetrate the sample and hybridize with the encoding probes. The length of the incubation step can depend on the type of sample, buffer reagents used, readout probe length, and other factors. For example, the incubation step can be between 1000 and 1500 seconds.

The system then supplies a series of buffers to the flow cell 110 via the multi-valve positioner 114 to prepare the sample for imaging. The series of buffers can include a wash buffer which can include reagents to displace unbound and excessive components which may interfere with the assay, such as an astringent reagent (e.g., formamide). The series of buffers can further include a hybridization buffer to control stringency and eliminate residual fluorescent material or autofluorescence of the sample. The series of buffers can further include an imaging buffer to prepare the sample and probes for imaging, such as performing oxygen scavenging (e.g., glucose oxidase). The combined incubation and buffer flow steps can take between 1200 and 1500 seconds, although other durations are possible depending on experimental conditions such as flow speed.

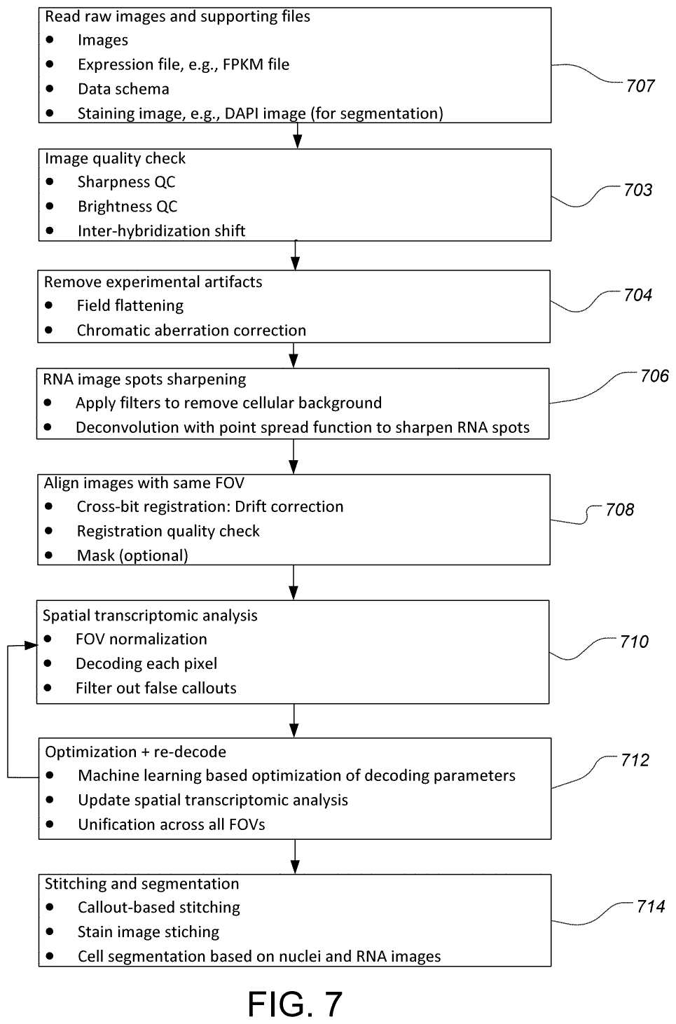

The system then performs an imaging step (204) in which all lateral fields-of-view (FOVs) of the sample are imaged, described further in FIG. 7. Briefly, the imaging includes all necessary steps to image each FOV across the number of color channels available to the system. For example, the system can include a light source 122 and emission filter wheel 128 for four color channels per FOV, though more can be considered. The light source 122 consecutively excites the fluorophores of the set of readout probes localized within the selected FOV while the filter wheel 128 allows for the collection of the emitted fluorescence to form a fluorescent image. In some implementations, the imaging system may be configured, e.g., with a color camera, to image multiple fluorophores of different emission wavelengths simultaneously. This captures the lateral and vertical position of the readout probes hybridized in the preceding step. The time required for imaging can vary based on sample size, illumination intensity, and fluorescence intensity. In general, imaging can take between 300 and 600 seconds.

The fluorophore of the hybridized readout probes are then photobleached (206). This begins by the multi-valve positioner 114 supplying a volume of bleaching buffer to the flow cell 110 to displace and purge the imaging buffer. The photobleaching includes bathing the sample 10 in the flow cell 110 with high intensity light to photochemically render the readout probes hybridized within the sample fluorophores permanently unable to fluoresce. The light source is chosen to correspond to the fluorophores used in combination with the readout probes. For example, a light source with a wavelength, or a broad spectrum light source filtered to the same, between 400 and 600 nm can be used. The power necessary to render the fluorophores unable to fluoresce can be between 100 and 400 mW. The time for a photobleaching step can vary depending on light source wavelength and power applied. In general, between 2 and 10 s is sufficient. However, some samples require longer times, and/or multiple rounds of photobleaching.

A single round of hybridization, buffer washes, image capture, and photobleaching can take between 50 and 70 m. The process can then be repeated (207) with additional rounds of hybridization, buffer washes, imaging, and photobleaching. For example, a mFISH experiment can include between 4 and 20 rounds of hybridization and mFISH imaging with unique readout probes used in each round. Higher numbers of rounds require correspondingly large time investments. As a result, increasing the number of readout bit calls so as to increase data depth in a given image pixel may be impractical or commercially prohibitive.

Disclosed herein is a method of increasing the number of bit readout calls (also known simply as “readout calls”, or “bit calls”) performed per round of hybridization. By introducing in situ switchable readout probes, the number of readout calls is improved within a round of hybridization and within a color channel. Assuming the number of color channels stays the same, the total number of readout calls can be increased. Alternatively, while more readout calls can be obtained from a given color channel, the number of color channels can be reduced while the total number of readout calls is maintained. This permits a user to select particular channels which give optimal performance for a given assay, and avoid others which are impacted by autofluorescence.

A readout call occurs during imaging when the light source 122 excites the fluorophores of the hybridized readout probes within the sample 10. The excited fluorophores return to the ground state and emit light of a given wavelength which is received by the microscope 120. An encoding probe having a bound readout probe with an emitting fluorophore can be considered an “on” bit. An encoding probe that does not have an emitting fluorophore can be considered an “off” bit. An encoding probe can fail to emit for reasons such as excitation light of improper wavelength for the fluorophore, localized quenching, or chemical- or photo-bleaching. Additionally, the bit state of an encoding probe can be switched during a switching step, turning an “on” bit to “off”, or vice versa. For example, a fluorophore can be cleaved from the readout probe, or the readout probe can be washed away, or a quencher can be cleaved so that it no longer quenches the fluorophore.

A switching step can include one or more means to switch the binary state of a bit from the previously imaged state. For example, if a bit is “on” in a first image, the switching step may switch the bit to “off” before consecutive images are taken in the different color channels. Conversely, if a bit is “off” in a first image, the switching step may switch the bit to “on” before consecutive images are taken in the different color channels. The techniques for switching are dependent on the construction of readout probes contained within the set of hybridized readout probes, and one or more switching steps can be performed in between collected images.

The fluorescence of a readout probe can be switched through one or more switching modalities that can include modification of the one or more fluorophores or modification of the targeting sequence 410. Examples of fluorophore modifications can include quenching or de-quenching of the fluorophore. Examples of targeting sequence 410 modification can include dissociation, cleavage, or competitive binding.

Fluorescence occurs when a fluorophore is excited at a particular wavelength and promoted to an excited state. The excited dye then emits light in returning to the ground state. Examples of fluorophores can include 7-AAD, Acridine Orange, Alexa Fluor® dyes, BFP (Blue Fluorescent Protein), GFP (Green Fluoresecent Protein), BODIPY® dyes, CFP (Cyan Fluorescent Protein), Cyanine-based dyes, DAPI, Ethidium bromide, Fluorescein-based dyes, Lucifer dyes, Oregon dyes, Rhodamine-based dyes, SYTO® dyes, Thiazole-based dyes, YFP (Yellow Fluorescent Protein), YOYO® dyes, ATTO dyes, or IRDye® dyes.

Fluorophore quenching refers to any process that decreases the emitted intensity of this process. A variety of molecular interactions can result in quenching. These include excited-state reactions, molecular rearrangements, energy transfer, ground-state complex formation, and collisional quenching. The quencher could be a single large molecule, ion, nanoparticle, or nanostructure. When a quencher is present, the excited fluorophore can return to the ground state by transferring its energy to the quencher, without the emission of light, while the quencher is promoted to its excited state. The quencher can then emit the energy in a non-detected wavelength or may release the energy in a non-radiative pathway. Quenchers can operate through Förster resonance energy transfer (FRET) or Dexter electron transfer (DEX) pathways both of which depend on the fluorophore and quencher being in close proximity, e.g., >10 nm. Without wishing to be bound by theory, the quencher selection depends on the emission-absorption spectral overlap with the fluorophore and the relative orientation of the donor and acceptor transition dipole moments. It should be understood that quencher selection depends on the fluorophore used in the readout probe.

Fluorescence modification using quenching mechanics can include the addition or removal of quencher molecules thereby switching the fluorescence on or off in a switching step. In some embodiments, one quencher molecule per fluorophore present in a readout probe is attached to a readout probe through a cleavage domain. An intact cleavage domain maintains the quencher within a distance that preserves the quenching (e.g., FRET, or DEX) pathways (e.g., >10 nm). The switching step then would include a means which cleaves the cleavage domain between the quencher and the fluorophore. Removal of the quencher turns the readout probe “off” bit to “on”.

An example of the addition of a quencher includes the addition of a quencher probe to the flow cell. A quencher probe includes a quencher attached to a nucleotide targeting sequence that specifically binds a portion of a hybridization region adjacent to a readout probe. The quencher probe targeting sequence can be designed such that when bound to the hybridization region, the quencher molecule is spaced a distance adjacent to the fluorophore of the readout probe in which quenching occurs (e.g., FRET, or DEX). The switching step would then include the addition of the quencher probe and sufficient incubation time between hybridization rounds. Addition of the quencher probe would switch the readout probe “on” bit to “off”. Examples of quenchers can include TAMRA, Black Berry Quencher-650, ECLIPSE™, DyQ® quenchers, Black Hole Quenchers®, QSY® quenchers, IRDye® quenchers, Iowa Black® FQ, Iowa Black® RQ, acrylamide, a dabcyl group, and any derivatives thereof.

In some embodiments, the fluorescence modification can include the use of FRET or DEX pairs of fluorophores to switch the readout calls. In a FRET or DEX pair, one of the fluorophores is the donor and the second fluorophore the acceptor. When in proximity, an excited donor fluorophore non-radiatively transfers excitation energy to a nearby acceptor fluorophore. The acceptor fluorophore then returns to the ground state by emitting light in a wavelength that the imaging system can detect. In this manner, the addition or removal of a FRET or DEX pair fluorophore switches the bit state of the first fluorophore. For example, a set of readout probes can include a first donor readout probe that does not emit in a wavelength detectable by the system. A second readout probe with an acceptor fluorophore can be added to the sample to change the fluorophore readout call from “off” to “on”.

Fluorescence modification using nucleotide modification can include the removal of a fluorophore from a readout probe. An example of removal of a fluorophore includes a fluorophore attached to the nucleotide sequence of a readout probe through a cleavable bond or short cleavable nucleotide sequence, e.g., >30 bp., thereby switching an “on” bit to an “off”. In some implementations, the cleavable bond or short cleavable nucleotide sequence can be within the readout probe nucleotide sequence. The switching step then would include a means which disrupts the bond between the quencher and the fluorophore, such as photocleaving, enzymatic cleaving, or chemical cleaving. Removal of the quencher turns the readout probe “off” bit to “on”.

Fluorescence modification can further include differential removal of one or more readout probes of a set of hybridized readout probes from the encoding probe. For example, the set of readout probes can include a first readout probe with a short targeting sequence (e.g., <20 bp) and a second readout probe with a longer targeting sequence (e.g., >40 bp). A buffer supplied to the flow cell after hybridization of the set of readout probes can include chemical reagents to release hybridized readout probes from the readout regions of the encoding probes. The buffer can be incubated with the sample 10 for a time sufficient to separate readout probes with short targeting sequences, and be purged before the readout probes when long targeting sequences dissociate. In this example, removal of readout probes with short targeting sequences turns those bits to “off” while readout probes with long targeting sequences remain “on”.

In another example, the targeting sequence of a readout probe can be designed to partially complement (e.g., non-specific, 30-70% basepair complementation) a hybridization region of an encoding probe. A buffer supplied to the flow cell after hybridization of the set of readout probes can use non-specific binding agents to competitively bind hybridized readout probes with partially complementary targeting sequences. In this example, competitive binding of readout probes with non-specific targeting sequences turns those bits to “off” while readout probes with specific targeting sequences remain “on”.

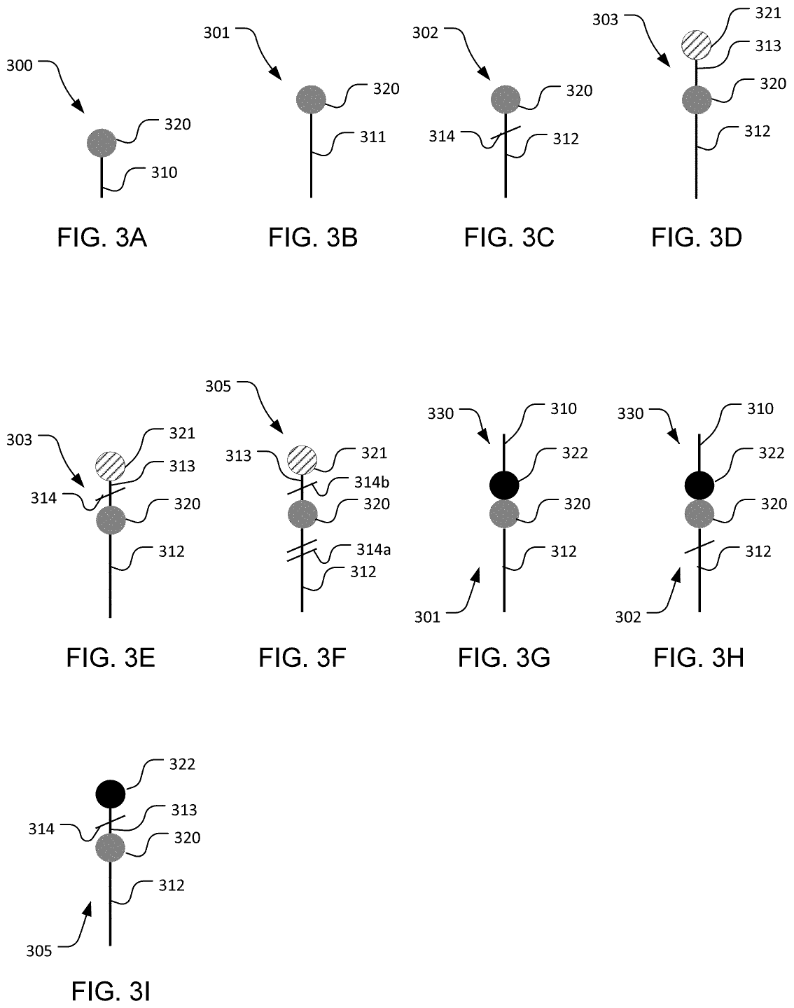

FIGS. 3A through 3I depict several examples of readout probe construction for use in a fluorescence switching step. As shown in FIG. 3A and as described above, a readout probe 300 includes at least one fluorophore 320 and nucleotide targeting sequence 310 complementary to one hybridization region of an encoding probe. The fluorophore 320 is stimulated by light of a first wavelength and emitting light of a second wavelength.

The targeting sequence 310 can include 8 to 100 bp of nucleotides, e.g., 15 to 45 bp of nucleotides. For example, the targeting sequence 310 of FIG. 3A depicts a first targeting sequence 310 and FIG. 3B depicts a readout probe 301 with a second targeting sequence 311 that is longer than the first targeting sequence 310 while sharing the same fluorophore 320.

In some embodiments, a readout probe 302 can include a targeting sequence 312 with one or more cleavage domains 314 as depicted in FIG. 3C. For example, the cleavage domain 314 can include a domain susceptible to photocleaving, enzymatic cleaving, or chemical cleaving. Photocleavable groups can include nitrobenzyl-based, carbonyl-based, or benzyl-based groups. Enzymatically cleavable sites include nucleotide sequences specifically targeted for cleavage by single-stranded nucleases such as the S1 or P1 endonuclease enzymes. Chemically cleavable sites include labile sites such as disulfide linkages (e.g., cleavable by mild reducing agent), ester linkages (e.g., cleavable with an acid, a base, or hydroxylamine), a peptide linkage (e.g., cleavable via a protease), or a phosphodiester linkage (e.g., cleavable via a nuclease). The cleavage domain within a targeting sequence 312 can be located at any point between the distal end to the proximal end in the targeting sequence 312, relative to the fluorophore 320. The targeting nucleotide sequence 312 can be of similar length to the nucleotide sequence 310, e.g., between 15 and 45 bp, or can be longer, e.g., of similar length to the nucleotide sequence 311.

In contrast, a “basic” readout probe as shown in FIGS. 3A and 3B can include just a single fluorophore 320, and need not include any specialized chemical structure that permits cleaving of the fluorophore 320 from the targeting sequence 310, 311.

As shown in FIGS. 3D, 3E and 3F, in some embodiments, the readout probe includes two or more fluorophores, each responsive to different excitation wavelengths. For example, as shown in FIG. 3D, readout probe 303 includes, in order, a targeting nucleotide sequence 312, a first fluorophore 320, a linker region 313, and a second fluorophore 321. As an example, the linker region can include a short (e.g., >15 bp) non-specific nucleotide sequence. In a further example, the linker region can be a chemical linkage such as a diethylene glycol linker. The readout probe can include a larger number of fluorophores, so long as each fluorophore has a different modification technique.

As shown in FIG. 3E, the linker region 313 can further include a cleavage site 314 as described herein. As a further example, FIG. 3F depicts readout probe 303 including, in order, a first targeting nucleotide sequence 312 with a first cleavage domain 314a, a first fluorophore 320, a second non-specific nucleotide sequence 313 with a second cleavage domain 314b, and a second fluorophore 321.

FIGS. 3G through 3I illustrate examples of readout probes using quenchers as the fluorescence modification switching modality. FIG. 3G depicts the readout probe 301 of FIG. 3B adjacent to quencher probe 330. Quencher probe 330 includes a targeting sequence 310 and a quencher 322. Examples of quenchers are described above. FIG. 3H shows the combination of the readout probe 302 with quencher probe 330. The combinations depicted in FIGS. 3F and 3G can be switched by the addition or removal of quencher probe 330.

FIG. 3I depicts readout probe 305 that includes, in order, a first targeting sequence 312, a fluorophore 320, a second non-specific nucleotide sequence 313 with a cleavage domain 314, and a quencher molecule 322. The readout probe 305 is constructed with the second non-specific nucleotide sequence 313 and quencher molecule 322 and can be switched from “off” to “on” by cleaving the cleavage site 314 in the second non-specific nucleotide sequence 313.

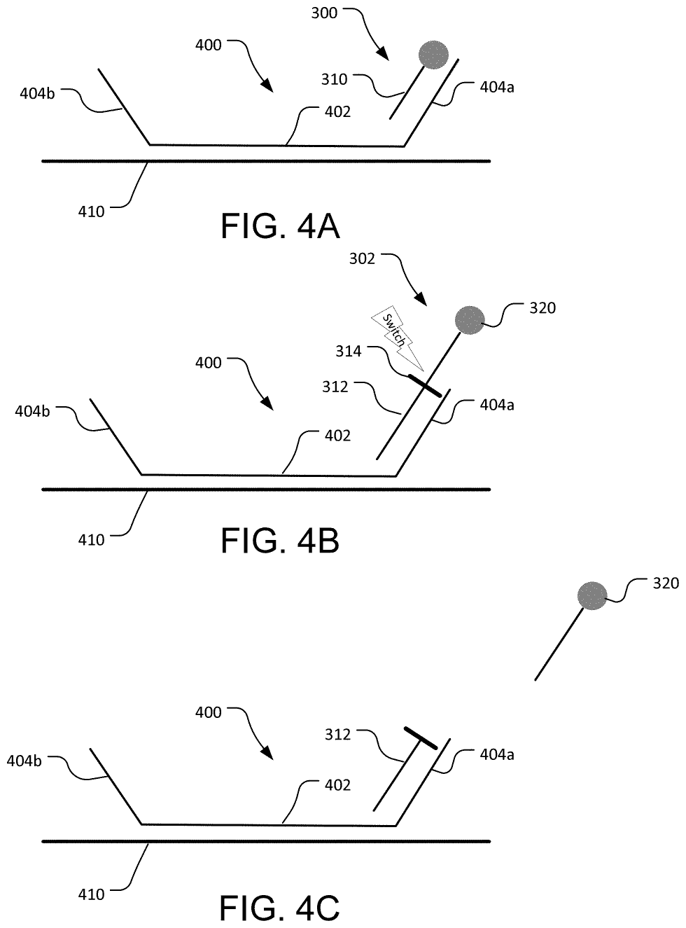

Using a switchable readout probe design, it is possible to gain further multiplexed information from within a single hybridization step. FIG. 4A depicts an exemplary encoding probe 400 and bound readout probe 300 used in a traditional mFISH system. The encoding probe 400 includes an encoding region 402, a first hybridization sequence 404a positioned at one end of the encoding region 402, and an optional second hybridization sequence 404b positioned at the opposite end of the encoding probe 400. The encoding region 402 is a sequence that is complimentary to and will bind specifically with a target sequence 410 within a sample 10.

The first hybridization sequence 404a and optional second hybridization sequence 404b are sequences complimentary to the targeting sequence 310 of the depicted readout probe 300. A readout probe 300 bound to the encoding probe 400 in this manner will function as an “on” bit during imaging, indicating that the target sequence 410 has been bound. However, in traditional mFISH this bit state is static until the sample 10 is photobleached, extinguishing all fluorescence.

A readout probe including a switching modality can modify the bit state of an encoding probe during a switching step. FIG. 4B depicts the encoding probe 400 bound to the target sequence 410 and a readout probe 302 including a targeting sequence 312 that includes a cleavage domain 314. In this configuration, the targeting sequence 312 is bound to the hybridization sequence 404a and the fluorophore is un-modified. The readout probe 300 will function as an “on” bit during imaging. The exemplary cleavage domain 314 can then be broken in a switching step (shown).

The results of the switching step are shown in FIG. 4C. The encoding probe 400 remains bound to the target sequence 410, and the targeting sequence 312 remains bound to the hybridization sequence 404a but the fluorophore 320 has been removed from the readout probe 302. The fluorophore 320 can be purged from the flow cell 110. In this manner, no fluorophore remains on the encoding probe 400 and the bit state has been switched to “off”.

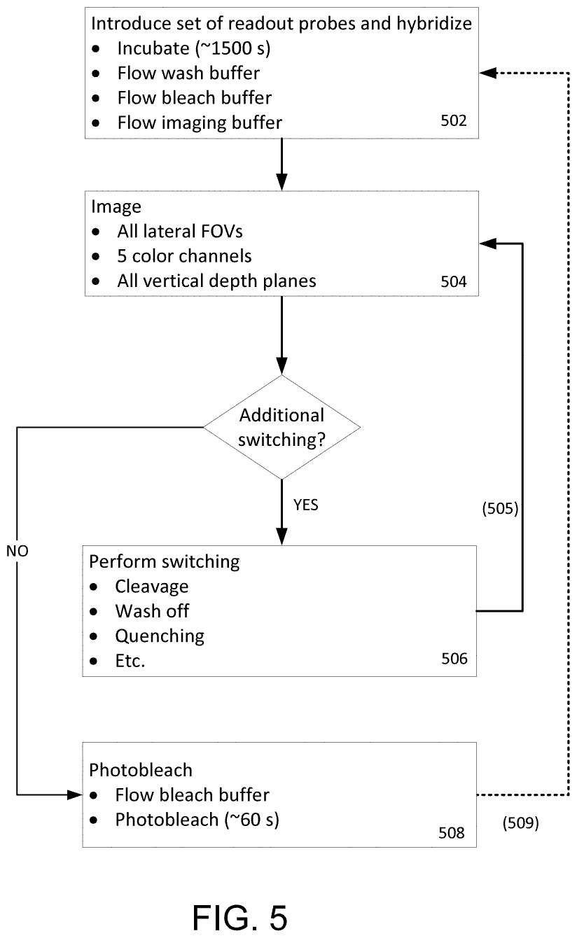

FIG. 5 shows an exemplary workflow using readout probes including switching modalities. The first round of hybridization of the readout probes (502) begins with the multi-valve positioner 114 supplying a buffer containing a first set of one or more switchable readout probes to the flow cell 110 containing the sample 10. As above, each set of switchable readout probes supplied to the flow cell 110 can include a number of unique fluorophores as color channels as the system is capable of imaging in a single image. As above, the readout probes are incubated with the sample for sufficient time to allow hybridization with the encoding probes. The wash, bleach, and imaging buffers are consecutively supplied, purging the previous volume from the flow cell 110 as in 302.

The imaging step (504) then occurs in which all lateral FOVs, and vertical positions therein, of the sample are imaged, as described above for FIGS. 1 and 3. Broadly, the light source 122 consecutively excites the fluorophores of the set of switchable readout probes localized within the selected vertical position and FOV while the filter wheel 128 allows for the collection of the emitted fluorescence to form a fluorescent image. This captures the lateral and vertical position of the switchable readout probes hybridized in the preceding step.

At least one round of switching occurs after all imaging for the sample has been collected a first time. The switching step modifies the fluorescence of a first group of switchable readout probes (506). Switching can be performed by cleavage, quenching, FRET/DEX, or washing. During this step, the bit state of a portion of readout probes can be switched depending on the switching modality present in the set of readout probes and the type of switching process. For example, a photocleavage step can switch the bit state of a readout probe including a photocleavage site but will not switch the bit state of a readout probe including a quenching probe. In general, the switching step leaves the fluorescence of at least a second group of readout probes unchanged. This second group of readout probes could be “basic” readout probes, or switchable readout probes that are not affected by the type of switching process use to switch the first group of readout probes.

As a result of the switching, a first set of bit states are switched (506) and an additional round of imaging (504) can be performed (505). The rounds of switching and additional imaging can be performed for as many switching modalities are included in the set of readout probes. In this manner, the bit state of a readout probe is an additional level of data multiplexing available within a single round of hybridization (502).

Once the switching steps have been completed, e.g., all of the available switching modalities have been used, the process can continue with a photobleaching (508) and repeated (509) rounds of hybridization (502) as described in FIG. 3.

Using switchable readout probes can allow for the collection of multiplexed information from a single switchable readout probe. Each readout probe targets a specific hybridization region within an encoding probe, and each encoding probe targets a specific nucleotide sequence within a sample. Switchable probes therefore allow multiplexed readout calls for a hybridization region and expand the bit depth of a gene code word bit.

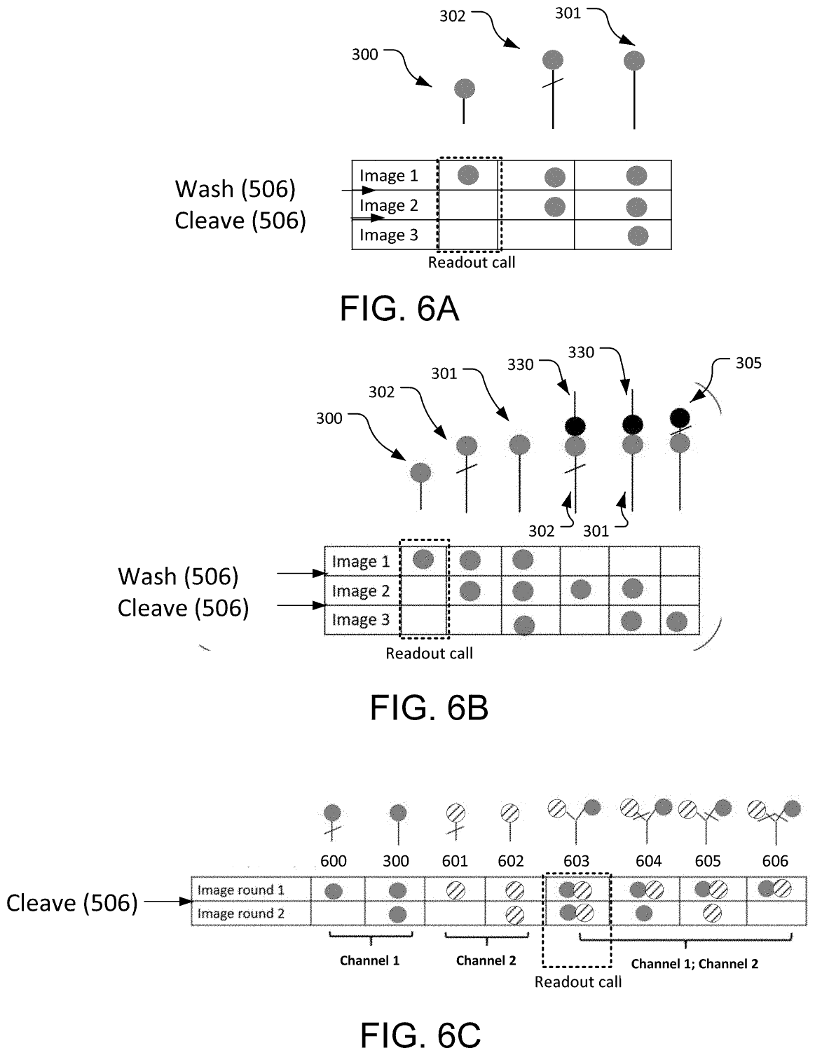

FIG. 6A depicts an exemplary set of three readout probes, each sharing a common fluorophore and having a unique targeting sequence designed to target three unique hybridization regions. When excited with a light source 122 and the emitted fluorescence collected to form a fluorescent image, the excited fluorophores produce an “on” bit in the positions corresponding to readout probes 300, 301, and 302, shown in the first row of the table of FIG. 6A.

A first switching step 506 is performed including a buffer wash step to unbind readout probe 300 which includes a short targeting sequence. Readout probes 302 and 301 have long targeting sequences and remain hybridized to their respective hybridization regions. A second image shows “on” bits and the position in the image that originally correlated to the first “on” bit switched to “off”.

A second switching step 506 is performed including a cleavage step to cleave the site in the targeting sequence of readout probe 302. Readout probe 301 does not have a cleavage site and will remain hybridized to the respective hybridization region while the fluorophore of readout probe 302 is purged. A third image will show one “on” bit and the positions in the image that originally correlated to the first and second “on” bit will now be switched to “off”.

The positional and bit state information in the three images can be correlated using image stacking methods described herein and each positional pixel that correlates to at least one “on” bit is termed a ‘readout call’, and is represented by columns of FIG. 6A. The readout calls (e.g., columns) of the three readout probe positions in the three images will each have a unique signature, corresponding to three respective hybridization regions using only a single color channel (e.g., fluorophore) and a single hybridization step.

The set of readout probes depicted in FIG. 6B includes the readout probes of FIG. 6A as well as two unique readout probes combinations, 302 and 301 hybridized adjacent to quencher probes 330, and readout probe 305 with a cleavable quencher molecule attached. The first image will show three “on” bits, similar to the first row of FIG. 6A. A wash step is performed (506), as in FIG. 6A, which purges readout probe 300 and quencher probes 330. Two new “on” bits will appear in the second image collected, and the position corresponding to readout probe 300 will be switched to “off”. A second switching step is performed, the exemplary cleavage step of FIG. 6A and a third image collected will show the second and fourth readout probe positions switched to “off” and a sixth readout probe position switched to “on” as the quencher molecule is cleaved from readout probe 305. The set of readout probes of FIG. 6B results in six unique readout calls within a single hybridization step and a single color channel.

The set of readout probes depicted in FIG. 6C includes eight exemplary readout probes. The readout probes of FIG. 6C are combinations of components including two fluorophores imaged using separate color channels, a targeting sequence, and a cleavage region. The table of FIG. 6C shows the eight possible readout calls from two images using these combinations. From the left, readout probe 600 includes a first fluorophore and a targeting sequence with the cleavage domain; readout probe 300 includes the first fluorophore and targeting sequence with no cleavage domain; readout probe 601 includes a second fluorophore and the targeting sequence including the cleavage domain; readout probe 602 includes the second fluorophore and targeting sequence with no cleavage domain; readout probe 603 includes both the first and second fluorophore linked to a targeting sequence with no cleavage domain; readout probe 604 includes both the first and second fluorophore, wherein the second fluorophore is linked to the targeting sequence via the cleavage domain; readout probe 605 includes both the first and second fluorophore, wherein the first fluorophore is linked to the targeting sequence via the cleavage domain; readout probe 606 includes both the first and second fluorophore, wherein both the first and second fluorophore are linked to the targeting sequence via the cleavage domain.

The first image will show eight “on” bits across two color channels, four single “on” bits and four doubled “on” bits. A switching step 506 is performed including a cleavage step to cleave the site of readout probes 600, 601, 603, 604, 605, and 606. A second image will then show readout call states of row two of the table of FIG. 6C, wherein the readout call of readout probes 300, 602, and 603 remaining “on”, readout probes 600, 601, and 606 being switched to “off”, and readout probes 604 and 605 being switched to distinguishable “on” states. This set of readout probes can achieve 8 distinct readout calls with a bit depth of two in a single round of hybridization and two images.

Returning to FIG. 1, the control system 140 is configured, i.e., by the control software and/or the workflow script, to acquire fluorescence images (also termed simply “collected images” or simply “images”) in loops in the following order (from innermost loop to outermost loop): z-axis, color channel, lateral position, switching, and reagent.

These loops may be represented by the following pseudocode:

for g = 1:N_hybridization

% multiple hybridizations
for h= 1:N_switch

% multiple switches
for f = 1:N_FOVs

% multiple lateral field-of-views
for c = 1:N_channels

% multiple color channels
for z = 1:N_planes

% multiple z planes
Acquire image(g, h, f, c, z);

end % end for z

end % end for c

end % end for f

end % end for h

end % end for g

For the z-axis loop, the control system 140 causes the stage 118 to step through multiple vertical positions. Because the vertical position of the stage 118 is controlled by a piezoelectric actuator, the time required to adjust positions is small and each step in this loop is extremely fast.

First, the sample can be sufficiently thick, e.g., a few microns, that multiple image planes through the sample may be desirable. For example, multiple layers of cells can be present, or even within a cell there may be a vertical variation in gene expression. Moreover, for thin samples, the vertical position of the focal plane may not be known in advance, e.g., due to thermal drift. In addition, the sample 10 may vertically drift within the flow cell 110. Imaging at multiple Z-axis positions can ensure most of the cells in a thick sample are covered, and can help identify the best focal position in a thin sample.

For the color channel loop, the control system 140 causes the light source 122 to step through different wavelengths of excitation light. For example, one of the laser modules is activated, the other laser modules are deactivated, and the emission filter wheel 128 is rotated to bring the appropriate filter into the optical path of the light between the sample 10 and the camera 134.

For the lateral position, the control system 140 causes the light source 122 to step through different lateral positions in order to obtain different fields of view (FOVs) of the sample. For example, at each step of the loop, the linear actuators supporting the stage 118 can be driven to shift the stage laterally. In some implementations, the control system 140 number of steps and lateral motion are selected such that the accumulated FOVs cover the entire sample 10. In some implementations, the lateral motion is selected such that FOVs partially overlap.

For the switching, the control system 140 causes the apparatus 100 to step through the available switching processes. For example, if the group of readout probes include a photolabile cleavage group 414, the control system 140 can cause the light source 122 to illuminate the flow cell 110 with light of a wavelength corresponding to the cleavage group and break the chemical linkage. In another example, if the group of readout probes include a chemically susceptible cleavage group 414, the control system 140 can cause the multi-valve positioner 114 to select a reagent 112a that targets the chemically susceptible cleavage group 414 and supply the reagent 112a to the flow cell 110. In a further example, if the sample contains a group of readout probes 400 or quencher probes 330 with a short targeting sequence 410, the control system 140 can cause the multi-valve positioner 114 to select a reagent 112a to wash away the readout 400 or quencher probes 330.

For the hybridization, the control system 140 causes the apparatus 100 to step through multiple different available reagents that include a set of one or more readout probes to be hybridized to the encoding probes within the sample 10. For example, at each step of the loop, the control system 140 can control the valve 114 to connect the flow cell 110 to the purge fluid 112b, cause the pump 116 to draw the purge fluid through the cell for a first period of time to purge the current reagent, then control the valve 114 to connect the flow cell 110 to a different new reagent, and then draw the new reagent through the cell for a second period of time sufficient for the probes in the new reagent to bind to the appropriate RNA sequences. Because some time is required to purge the flow cell and for the probes in the new reagent to bind, the time required to adjust reagents in is the longest, as compared to adjusting the lateral position, color channel or z-axis.

As a result, a fluorescence image is acquired for each combination of possible values for the z-axis, color channel (excitation wavelength), lateral FOV, switching, and reagent. Because the innermost loop has the fastest adjustment time, and the successively surrounding loops are of successively slower adjustment time, this configuration provides the most time efficient technique to acquire the images for the combination of values for these parameters.

A data processing system 150 is used to process the images and determine gene expression to generate the spatial transcriptomic data. At a minimum, the data processing system 150 includes a data processing device 152, e.g., one or more processors controlled by software stored on a computer readable medium, and a local storage device 154, e.g., non-volatile computer readable media, that receives the images acquired by the camera 134. For example, the data processing device 152 can be a workstation with GPU processors or FPGA boards installed. The data processing system 150 can also be connected through a network to remote storage 156, e.g., through the Internet to cloud storage.

In some implementations, the data processing system 150 performs on-the-fly image processing as the images are received. In particular, while data acquisition is in progress, the data processing device 152 can perform image pre-processing steps, such as filtering and deconvolution, that can be performed on the image data in the storage device 154 but which do not require the entire data set. Because filtering and deconvolution are a major bottleneck in the data processing pipeline, pre-processing as image acquisition is occurring can significantly shorten the offline processing time and thus improve the throughput.

FIG. 7 illustrates a flow chart of a method of data processing in which the processing is performed after all of the images have been acquired. The process begins with the system receiving the raw image files and supporting files (step 702). In particular, the data processing system can receive the full set of raw images from the camera, e.g., an image for each combination of possible values for the z-axis, color channel (excitation wavelength), lateral FOV, and reagent.

In addition, the data processing system can receive a reference expression file, e.g., a FPKM (fragments per kilobase of sequence per million mapped reads) file, a data schema, and one or more stain images, e.g., DAPI images. The reference expression file can be used to cross-check between traditional sequence results and the mFISH results.

The image files received from the camera can optionally include metadata, the hardware parameter values (such as stage positions, pixel sizes, excitation channels, etc.) at which the image was taken. The data schema provides a rule for ordering the images based on the hardware parameters so that the images are placed into one or more image stacks in the appropriate order. If metadata is not included, the data schema can associate an order of the images with the values for the z-axis, color channel, lateral FOV and reagent used to generate that image.

The stain images will be presented to the user with the transcriptomic information overlaid.

The collected images can be subjected to one or more quality metrics (step 703) before more intensive processing in order to screen out images of insufficient quality. Only images that meet the quality metric(s) are passed on for further processing. This can significantly reduce processing load on the data processing system. Examples of quality metrics include image sharpness, image brightness, and inter-hybridization shift, e.g., as detected by phase correlation.

Next, each image is processed to remove experimental artifacts (step 704). Since each RNA molecule will be hybridized multiple times with probes at different excitation channels, a strict alignment across the multi-channel, multi-round image stack is beneficial for revealing RNA identities over the whole FOV. Removing the experimental artifacts can include field flattening and/or chromatic aberration correction. In some implementations, the field flattening is performed before the chromatic aberration correction.

Each image is processed to provide RNA image spot sharpening (step 706). RNA image spot sharpening can include applying filters to remove cellular background and/or deconvolution with point spread function to sharpen RNA spots.

The images having the same FOV are registered to align the features, e.g., the cells or cell organelles, therein (step 708). To accurately identify RNA species in the image sequences, features in different rounds of images are aligned, e.g., to sub-pixel precision. However, since an mFISH sample is imaged in aqueous phase and moved around by a motorized stage, sample drifts and stage drifts through an hours-long imaging process can transform into image feature shifts, which can undermine the transcriptomic analysis if left unaddressed. In other words, even assuming precise repeatable alignment of the fluorescence microscope to the flow cell or support, the sample may no longer be in the same location in the later image, which can introduce errors into decoding or simply make decoding impossible.

One conventional technique to register images is to place fiducial markers, e.g., fluorescent beads, within the carrier material on the slide. In general, the sample and the fiducial marker beads will move approximately in unison. These beads can be identified in the image based on their size and shape. Comparison of the positions of the beads permits registration of the two images, e.g., calculation of an affine transformation.

A registration quality check can be performed after registration. If properly registered, the bright points in each image should overlap so that the total brightness is increased.

Optionally, after registration, a mask can be calculated for each collected image. In brief, the intensity value for each pixel is compared to a threshold value. A corresponding pixel in the mask is set to 1 if the intensity value is above the threshold, and set to 0 if the intensity value is below the threshold. The threshold value can be an empirically determined predetermined value, or can be calculated from the intensity values in the image. In general, the mask can correspond to the location of cells within the sample; spaces between cells should not fluoresce and should have a low intensity.

The data processing apparatus can now perform optimization and re-decoding (step 712). The optimization can include machine-learning based optimization of the decoding parameters, followed by returning to step 710 with updated decoding parameters in order to update the spatial transcriptomic analysis. This cycle can be repeated until the decoding parameters have stabilized.

The optimization of the decoding parameters will use a merit function, e.g., a FPKM/TPM correlation, spatial correlation, or confidence ratio. Parameters that can be included as variables in the merit function include the shape (e.g., start and end of frequency range, etc.) of the filters used to remove cellular background, the numerical aperture value for the point spread function used to sharpen the RNA spots, the quantile boundary Q used in normalization of the FOV, the bit ratio threshold THBR, the bit brightness threshold THBB (or the quantiles used to determine the bit ratio threshold THBR and bit brightness threshold THBB), and/or the maximum distance D1max at which at which a pixel word can be considered to match a code word.

This merit function may be an effectively discontinuous function, so a conventional gradient following algorithm may be insufficient to identify the optimal parameter values. A machine learning model can be used to converge on parameter values.

Next, the data processing apparatus can perform unification of the parameter values across all FOVs. Because each FOV is processed individually, each field can experience different normalization, thresholding, and filtering settings. As a result, a high contrast image can result in a histogram with variation that causes false positive callouts in quiet areas. The result of unification is that all FOVs use the same parameter values. This can significantly remove callouts from background noise in quiet areas, and can provide a clear and unbiased spatial pattern in a large sample area.

A variety of approaches are possible to select a parameter value that will be used across all FOVs. One option is to simply pick a predetermined FOV, e.g., the first measured FOV or a FOV near the center of the sample, and use the parameter value for that predetermined FOV. Another option is to average the values for the parameter across multiple FOVs and then use the averaged value. Another option is to determine which FOV resulted in the best fit between its pixel words and tagged code words. For example, a FOV with the smallest average distance d(p,b1) between the tagged code words and the pixel words for those code words can be determined and then selected.

The data processing apparatus can now perform stitching and segmentation (step 714). Stitching combines multiple FOVs into a single image. Stitching can be performed using a variety of techniques.

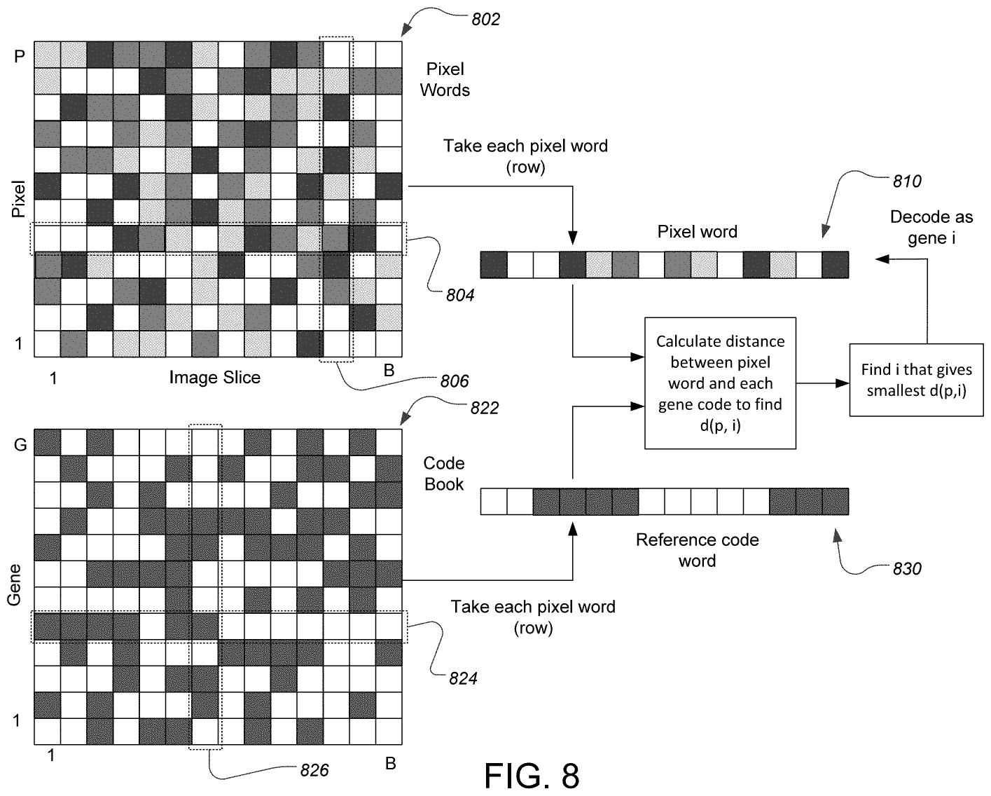

Decoding is explained with reference to FIG. 8. Aligned images for a particular FOV can be considered as a stack that includes multiple image layers, with each image layer being X by Y pixels, e.g., 2048×2048 pixels. The number of image layers, B, depends on the combination of the number of color channels (e.g., number of excitation wavelengths), number of switching states, and number of hybridizations (e.g., number of reactants), e.g., B=N_hybridization*N_switch*N_channels. In short, each color channel from each image can provide an image slice.

After normalization, this image stack can be evaluated as a 2-D matrix 802 of pixel words. The matrix 802 can have P rows 804, where P=X*Y, and B columns 806, where B is the number of images in the stack for a given FOV, e.g., N_hybridization*N_switch*N_channels. Each row 804 corresponds to one of the pixels (the same pixel across the multiple images in the stack), and the values from the row 804 provide a pixel word 810. Each column 806 provides one of the values in the word 810, i.e., the intensity value from the image layer for that pixel. As noted above, the values can be normalized, e.g., vary between 0 and IMAX. Different intensity values are represented in FIG. 8 as different degrees of shading of the respective cells.

If all the pixels are passed to the decoding step, then all P words will be processed as described below. However, pixels outside cell boundaries can be screened out by the 2-D masks (see FIG. 4B above) and not processed. As result, computational load can be significantly reduced in the following analysis.

The data processing system 150 stores a code book 822 that is used to decode the image data to identify the gene expressed at the particular pixel. The code book 822 includes multiple reference code words, each reference code word associated with a particular analyte, e.g., gene. As shown in FIG. 8, the code book 822 can be represented as a 2D matrix with G rows 824, where G is the number of code words, e.g., the number of genes (although the same gene could be represented by multiple code words), and B columns 826. Each row 824 corresponds to one of the reference code words 830, and each column 806 provides one of the values in the reference code word 830, as established by prior calibration and testing of known genes. For each column, the values in the reference code word 830 can be binary, i.e., “on” or “off”. For example, each value can be either 0 or IMAX, e.g., 1. The on and off values are represented in FIG. 8 by light and dark shading of respective cells. Thus, each bit in the reference code word can correspond to one of the image slices.

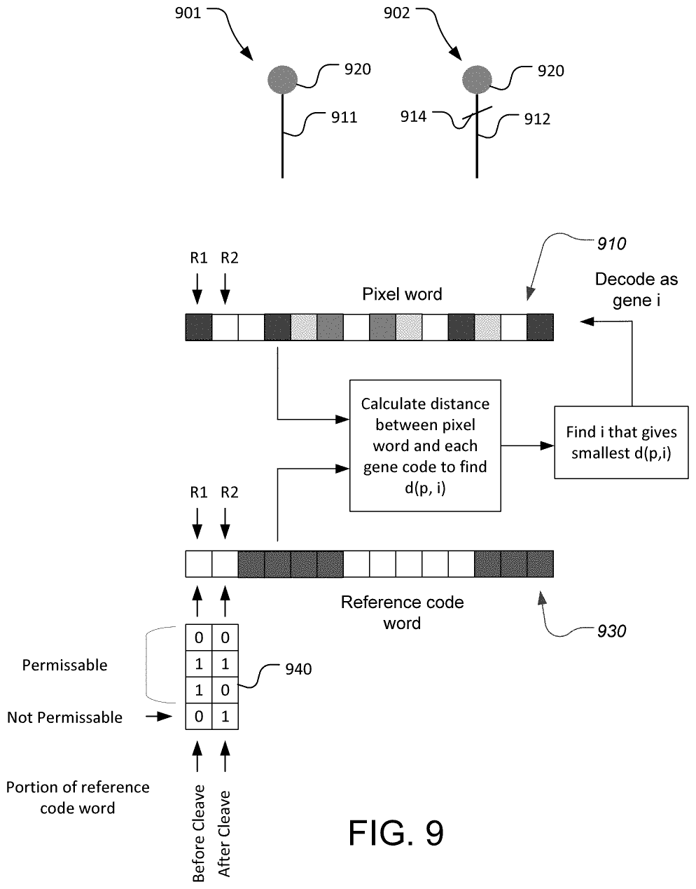

Depending on the combination of probes that are used in the process, some combinations of bit values can be expected to never occur; the reference code word would not use such combinations of bit values. An example of this is discussed further below with respect to FIG. 9.

Continuing with FIG. 8, for each pixel to be decoded, a distance d(p,i) is calculated between the pixel word 810 and each reference code word 830. For example, the distance between the pixel word 810 and reference code word 830 can be calculated as a Euclidean distance, e.g., a sum of squared differences between each value in the pixel word and the corresponding value in the reference code word. This calculation can be expressed as:

$$d ( p , i ) = {\sum }_{x = 1}^{B} {( {I}_{p , x} - {C}_{i , x} )}^{2}$$

where Ip,x are the values from the matrix 802 of pixel words and Ci,x are the values from the matrix 822 of reference code words. Other metrics, e.g., sum of absolute value of differences, cosine angle, correlation, etc., can be used instead of an Euclidean distance.

Once the distance values for each code word are calculated for a given pixel, the smallest distance value is determined, and the code word that provides that smallest distance value is selected as the best matching code word. Stated differently, the data processing apparatus determines min (d(p,1), d(p,2), . . . d(p,B)), and determines the value b as the value for i (between 1 and B) that provided the minimum. The analyte, e.g., gene, corresponding to that best matching code word is determined, e.g., from a lookup table that associates code words with analytes, and the pixel is tagged as expressing the analyte, e.g., gene.

An indication that a gene is expressed at a certain coordinate in the combined fluorescence image (as determined from the coordinate in the FOV and the horizontal and vertical shift for that FOV) can be added, e.g., as metadata. This indication can be termed a “callout.” Returning to FIG. 7, the data processing apparatus can filter out false callouts. One technique to filter out false callouts is to discard tags where the distance value d(p,b) that indicated expression of a gene is greater than a threshold value, e.g., if d(p,b)>D1MAX.

Referring now to FIG. 9, an example of the method of decoding using two readout probes is illustrated. Two probes, readout probe 901 and readout probe 902, are shown each having a fluorophore 920. Readout probe 901 includes a first targeting sequence 911 and readout probe 902 includes second targeting sequence 912 and a cleavage site 914. For example, readout probe 901 can be the probe from FIG. 3B, and readout probe 902 can be the probe from FIG. 3C.

FIG. 9 shows a pixel word 910 from a series of example image slices, such as pixel word 810, and a code word 930 drawn from an example code book, such as reference code word 830 from the matrix 822.

Within a single round of hybridization including a single cleavage step, an image is taken before cleaving, cleavage site 914 is cleaved, and another image is taken after cleaving. For a particular pixel in the image stack, this provides values R1 and R2 of pixel word 910 corresponding to the two image slice. Thus, the color channel corresponding the fluorophore 920 can provide two values for a pixel, R1 and R2. The code word 930 includes first bit corresponding to the image slice for R1 before cleavage and a second bit corresponding to the image slice for R2 from after cleavage, both within the same the hybridization round.

Depending on the composition of the probes used in the hybridization round, some combinations of bits will not be permissible in the code word. For example, for the combination of readout probe 901 and readout probe 902 a matrix 940 of all binary combinations is shown. The top-most combinations of 00, 11, and 10 are permissible from the pre-cleaving and post-cleavage imaging. However, the combination of 01 is not permissible because in this particular combination of probes there are no probes with fluorophores that are activated only after a treatment step.

As described herein, the steps for computing the distance between the pixel word 910 and the code word 930 and then decoding the pixel word 910 are the same.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method of fluorescent in-situ hybridization imaging of a sample, comprising:
exposing the sample to a plurality of encoding probes,
each encoding probe of the plurality of encoding probes having an encoding portion that targets a nucleotide sequence in the sample and a hybridization portion;
exposing the sample to a first plurality of first readout probes and a second plurality of second readout probes,
each first readout probe of the first plurality of first readout probes having a fluorophore to emit light at a first wavelength range and a first targeting portion that binds to a first hybridization sequence in a first encoding probe of the plurality of encoding probes,
each second readout probe of the second plurality of second readout probes having a fluorophore to emit light at the first wavelength range and a second targeting portion that binds to a second hybridization sequence in a second encoding probe of the plurality of encoding probes;
obtaining a first image of the sample with the first encoding probes and the second encoding probes having the first readout probes and the second readout probes, respectively, bound thereto so as to emit light at the first wavelength range;
treating the sample so as to modify the second readout probes on the second plurality of second encoding probes; and
obtaining a second image of the sample with the first encoding probes having the first readout probes bound thereto so as to emit light at the first wavelength range and the second encoding probes substantially not emitting light in at the first wavelength range.

2. The method of claim 1, wherein treating the sample deactivates the fluorophore of the second readout probe and the first image is obtained before the second image is obtained.

3. The method of claim 2, wherein the second readout probe includes a cleaving region between the fluorophore and the second targeting portion, and treating the sample comprises cleaving the cleaving region.

4. The method of claim 3, wherein cleaving the cleaving region comprises photocleaving and/or chemical cleaving.

5. The method of claim 2, wherein the second targeting portion of the second readout probes is shorter than the first targeting portion of the first readout probes, and treating the sample comprises washing the sample to preferentially wash away the second readout probes.

6. The method of claim 1, wherein treating the sample activates the fluorophore of the second readout probe and the first image is obtained after the second image is obtained.

7. The method of claim 6, comprising exposing the sample to a first plurality of quencher probes, each quencher probe of the plurality of quencher probes having a quencher and a third targeting portion that binds to the second hybridization sequence at a position such that the quencher suppresses emission of light by the fluorophore of the second readout probe.

8. The method of claim 7, wherein treating the sample comprises washing the sample to preferentially wash away the quencher probes while the second readout probes remain bound to the second hybridization sequence.

9. The method of claim 6, wherein the second readout probe includes a quencher and a cleaving region between the fluorophore and the quencher, and treating the sample comprises cleaving the cleaving region.

10. The method of claim 1, wherein the second readout probe has a second fluorophore to emit light at a second wavelength range.

11. The method of claim 10, comprising exposing the sample to a third plurality of third read out probes, each third readout probe of the third plurality of third readout probes having a fluorophore to emit light at the second wavelength range and a third targeting portion that binds to a third hybridization sequence in a third encoding probe of the plurality of encoding probes.

12. The method of claim 11, comprising treating the sample so as to modify the second readout probes on the second plurality of second encoding probes so as to activate or deactivate the second fluorophore.

13. The method of claim 12, comprising:
obtaining a third image of the sample with the second encoding probes and the third encoding probes having the second readout probes and the third readout probes, respectively, bound thereto so as to emit light at the second wavelength range,
obtaining a fourth image of the sample with the third encoding probes having the third readout probes bound thereto so as to emit light at the second wavelength range and the second encoding probes substantially not emitting light at the second wavelength range.

14. The method of claim 1, comprising exposing the sample to a third plurality of third readout probes, each third readout probe of the third plurality of third readout probes having a fluorophore to emit light at the first wavelength range and a third targeting portion that binds to a third hybridization sequence in a third encoding probe of the plurality of encoding probes.

15. The method of claim 14, comprising obtaining the first image of the sample with the first encoding probes, the second encoding probes and third encoding probes having the first readout probes, second readout probes and third readout probes, respectively, bound thereto so as to emit light at the first wavelength range.

16. The method of claim 15, comprising:
treating the sample so as to modify the third readout probes on the third plurality of third encoding probes; and
obtaining a third image of the sample with the first encoding probes having the first readout probes bound thereto so as to emit light at the first wavelength range and the third encoding probes substantially not emitting light at the first wavelength range.

17. The method of claim 16, wherein for the second image of the sample, the third encoding probes have the third readout probes bound thereto so as to emit light at the first wavelength range.

18. The method of claim 17, wherein for the third image of the sample, the second encoding probes substantially do not emit light at the first wavelength.

19. The method of claim 1, wherein treating the sample so as to modify a fluorescence comprises treating the sample with chemical cleavage, chemical quenching, or unbinding from the first readout region.

20. The method of claim 1, wherein the fluorophore comprises a fluorescent material or a phosphorescent material.

21. A method of fluorescent in-situ hybridization imaging, comprising:
exposing the sample to a first plurality of first readout probes and a second plurality of second readout probes,
each first readout probe of the first plurality of first readout probes having a fluorophore to emit light at a first wavelength range and a first targeting portion that binds to a first nucleotide sequence in the sample,
each second readout probe of the second plurality of second readout probes having a fluorophore to emit light at the first wavelength range and a second targeting portion that binds to a second nucleotide sequence in the sample;
obtaining a first image of the sample with first readout probes and the second readout probes bound to the first nucleotide sequence and the second nucleotide sequence, respectively, so as to emit light at the first wavelength range;
treating the sample so as to modify the second readout probes; and
obtaining a second image of the sample with the first readout probes bound to the first nucleotide sequence so as to emit light at the first wavelength range and the second nucleotide sequence substantially not emitting light at the first wavelength range.

22. A method of fluorescent in-situ hybridization imaging, comprising:
exposing the sample to a plurality of encoding probes,
each encoding probe of the plurality of encoding probes having an encoding portion that targets an analyte in the sample and a hybridization portion;
exposing the sample to a first plurality of first readout probes and a second plurality of second readout probes,
each first readout probe of the first plurality of first readout probes having a fluorophore to emit light at a first wavelength range and a first targeting portion that binds to a first hybridization sequence in a first encoding probe of the plurality of encoding probes,
each second readout probe of the second plurality of second readout probes having a fluorophore to emit light at the first wavelength range and a second targeting portion that binds to a second hybridization sequence in a second encoding probe of the plurality of encoding probes;
obtaining a first image of the sample with the first encoding probes and the second encoding probes having the first readout probes and the second readout probes, respectively, bound thereto so as to emit light at the first wavelength range;
treating the sample so as to modify the second readout probes on the second plurality of second encoding probes; and
obtaining a second image of the sample with the first encoding probes having the first readout probes bound thereto so as to emit light at the first wavelength range and the second encoding probes substantially not emitting light in at the first wavelength range.

23. A method of fluorescent in-situ hybridization imaging, comprising:
exposing the sample to a first plurality of first readout probes and a second plurality of second readout probes,
each first readout probe of the first plurality of first readout probes having a fluorophore to emit light at a first wavelength range and a first targeting portion that binds to a first analyte in the sample,
each second readout probe of the second plurality of second readout probes having a fluorophore to emit light at the first wavelength range and a second targeting portion that binds to a second analyte in the sample;
obtaining a first image of the sample with first readout probes and the second readout probes bound to the first analyte and the second analyte, respectively, so as to emit light at the first wavelength range;
treating the sample so as to modify the second readout probes; and
obtaining a second image of the sample with the first readout probes bound to the first analyte so as to emit light at the first wavelength range and the second analyte substantially not emitting light at the first wavelength range.
